(19) United States
(12) Patent Application Publication
(10) Pub. No.: US 2023/0383233 A1
(43) Pub. Date: Nov. 30, 2023

(54) APPARATUS FOR CONTROLLED ENVIRONMENT ESTABLISHMENT, MAINTENANCE, AND SAMPLING

(71) Applicant: BIOCONSORTIA, INC., DAVIS, CA (US)

(72) Inventors: NICHOLAS LITTLE, DAVIS, CA (US); DONALD GIBSON, DAVIS, CA (US)

(73) Assignee: BIOCONSORTIA, INC., DAVIS, CA (US)

(21) Appl. No.: 18/305,755

(22) Filed: Apr. 24, 2023

Related U.S. Application Data
(60) Provisional application No. 63/345,263, filed on May 24, 2022.

(51) Int. Cl. C12M 1/34; C12M 1/24; C12M 1/107; C12M 1/00; C12M 1/26

(52) U.S. Cl. CPC C12M 41/46; C12M 23/08; C12M 29/08; C12M 23/38; C12M 33/07; C12M 21/04

(57) ABSTRACT

Provided are improved compositions and methods for establishing, maintaining, and testing a sample in a controlled environment. The improved apparatus allows introduction and extraction of various desired compositions to and from the sample, while maintaining a sealed controlled environment to minimize experimental variability and other repercussions of system perturbation.

### CROSS-REFERENCE TO RELATED APPLICATIONS

This application claims the benefit under 35 U.S.C. 119(e) of U.S. Provisional Patent Application Ser. No. 63/345263 filed 24 May 2022, herein incorporated by reference in its entirety.

### FIELD

The instant disclosure relates to methods and apparatus for testing samples in controlled environments, specifically an improved design for sample collection, maintenance, and composition introduction and extraction.

### BACKGROUND

Control of environmental variables is an important aspect of experimental design, and testing within a stable environment can be complicated by repeated sample access. Existing commercially-available controlled environment systems are expensive, cumbersome, and lack the flexibility for application to a variety of situations.

For example, traditional testing of plant nitrogen fixation involves the use of glass mason jars with stoppers, which have distinct disadvantages, including the inability to maintain a consistent anaerobic state. Additionally, although those may be used in an anaerobic testing chamber for a limited period (not truly air-tight unless heated and sealed, so doesn't maintain anaerobicity with the concomitant ability for sample extraction and testing), the jars themselves must be placed in an anaerobic chamber for testing, rending the chamber equipment dirty and contaminated, with no good way to clean and sterilize the chamber between samples.

Thus, there is a need in the art for an improved apparatus that can accommodate a sample and allow for composition extraction without disturbance of the sample environment, for example for periodic or continuous assay. Although the examples provided herein describe testing of gases from plants, the invention finds use in a variety of fields that benefit from flexible controlled environment testing, such as but not limited to pharmaceuticals, tissue culture, microbial culture, and plant culture, as well as fields not involving living organisms or parts thereof, for example but not limited to chemical synthesis, distillation, inorganic matter testing, wastewater treatment systems, and the like.

### SUMMARY

The instant disclosure provides an improved design for samples, that minimizes handling and disturbance of the native environment. Broadly, it is contemplated that any sample for which a local environment must be maintained, and optionally sampled therefrom with minimal handling and disturbance, will benefit from the improved designs described herein. In some aspects, a sample is placed in the disclosed apparatus, and placed under a desired condition. A composition is extracted from the closed environment, for analysis or other use.

In some aspects, the system is closed but allows for exchange of gases and/or liquids.

In some aspects, sampling from the headspace of the apparatus is possible.

In some aspects, production and capture of Volatile Organic Compounds (VOCs) may be accomplished.

In some aspects, the improved apparatus may be hooked up to analytical equipment. In some aspects, the analytical equipment comprises High Performance Liquid Chromatography (HPLC) and/or Gas Chromatography (GC).

In some aspects, a volume of a sample may be extracted.

In some aspects, a living organism or part(s) thereof may be placed in the improved apparatus, to assess the consumption and/or production of any one or more compositions. In some aspects, the living organism or part(s) thereof are subjected to one or more specific conditions and/or stressors, to evaluate production and/or consumption of one or more compositions.

In some aspects, a plurality of living organisms or part(s) thereof may be assembled in a plurality of the improved apparatus. In some aspects, the assembly is in parallel. In some aspects, the assembly is in series. In some aspects, a Luer-lock system can create a pigtail mass gas exchange (pressure, etc.) system as long as tubing is exactly the same length, the same pressure will be applied.

In some aspects, a plurality of inputs is provided to the sample in the improved apparatus.

In some aspects, plants treated with microbes are placed in the apparatus for nitrogen fixation assessment

In some aspects, microbial cultures may be periodically or continuously introduced into the apparatus, which may optionally further comprise one or more plants.

In some aspects, the apparatus may be designed to use a standard-size container opening, for flexibility of transfer of the cap. In some aspects, this facilitates scale-up.

In some aspects, the apparatus facilitates field-expedient experiments. Plants or other items or organisms may be sourced directly and put into the bottle. Different conditions, such as the introduction of specific compositions such as gases, may be directly applied in the field.

In some aspects, the improved apparatus can operate under different environmental conditions, different gas pressures, and/or different organism interactions (e.g., plant-microbe).

In some aspects, the improved apparatus is part of a bioreactor or battery system for production of certain compositions or energy.

### BRIEF DESCRIPTION OF THE DRAWINGS AND THE SEQUENCE LISTIG

The disclosure can be more fully understood from the following detailed description and the accompanying drawings, which form a part of this application.

FIG. 1A shows an inferior glass jar sample container that is used commonly for material collection, maintenance, and sample extraction.

FIG. 1B shows one aspect of the improved apparatus disclosed and described herein.

FIG. 2 depicts a schematic of one aspect of the improved apparatus disclosed and described herein. The parts are labeled as follows: 1=sample container (e.g., bottle, jar, etc.) with a threaded opening; 2=container opening, which may optionally be threaded to accommodate an analogously-threaded cap; 3=container cap, which may further comprise threads that allow tight fitting against 2; 4=tubing that fits into the orifices of 5A; 5A=orifices in the cap of 3 that accommodate the tubing of 4; 5B=sealant for the openings created by the orifices of 5A; 6=valve (e.g., ballcock) that allows flow and/or cessation of flow from the inlet of the arrow indicated by “A”; 7=tubing that connects to the inlet of arrow “A”; 8=stopper with closeable access for a sampling instrument for outlet as indicated by the outlet arrow of “B”; “A”=inlet; “B”=outlet; 9=sample for analysis or other use.

FIG. 3 shows an example process for sample collection and preparation.

FIG. 4 shows an example for preparing samples (e.g., seeds) for microbial application, germination, and testing in the improved apparatus described herein.

FIG. 5 shows ethylene production results for two different designs of sample input (bag germinated seeds, jar germinated seeds).

### DETAILED DESCRIPTION

Provided are compositions and methods for an improved apparatus for sample collection, maintenance, and/or testing within controlled conditions.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

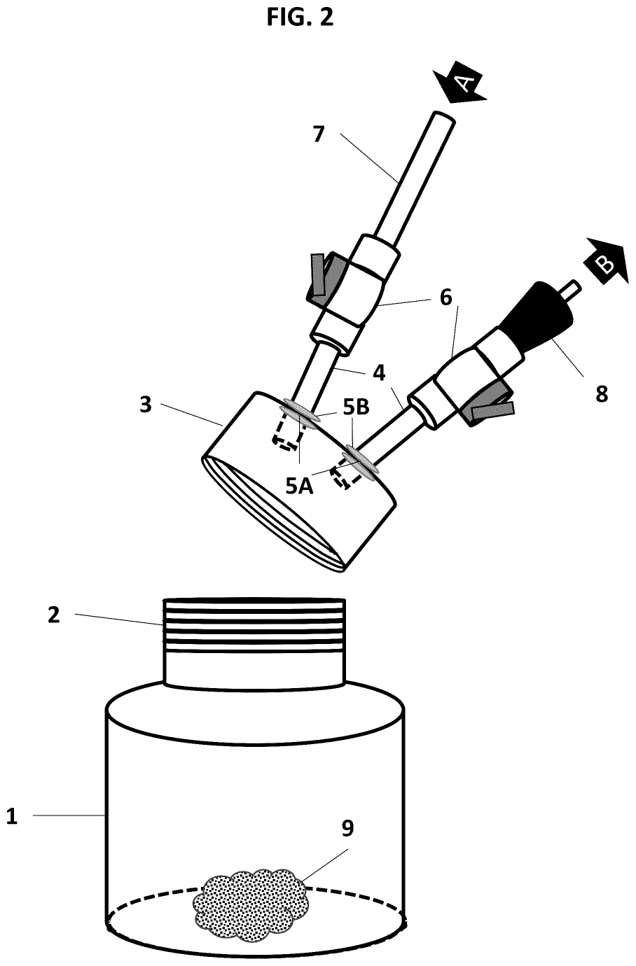

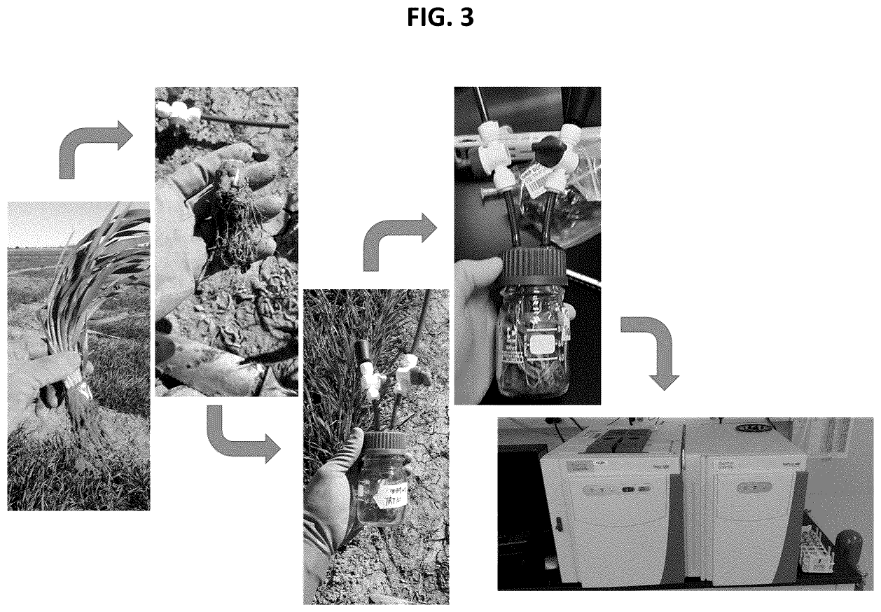

The parts of the system are depicted in FIG. 2.

The sample container (1) may be of any shape, size, or design, provided that it has a closed body and has an opening that accommodates a lid that is capable of providing a seal. The opening, and its corresponding lid, may be unthreaded, if there is a mechanism for friction or vacuum seal. Alternatively, the container and its corresponding lid may be synergistically threaded (2). The composition of the container may be the same or different than the composition of the lid. Depending on the type of analysis or sample requirement, the container may be of a material such as glass, plastic, metal, stone, or a synthetic polymer. In some cases, it may be desirable to control for the amount or wavelength of light to which the sample is exposed; therefore, different thicknesses, types, and/or colors of container material are contemplated for such uses. The sample container may be of a material suited for cleaning and/or sterilization, so that it may be reused. Alternatively, the sample container may be of a disposable material. The only requirement for the sample container is that it be capable of accommodating a lid that provides an adequate seal for the inlet and outlet lines that are attached thereto.

The lid (3) comprises two orifices, which may be placed on the top, or one or both of the orifices may be placed on a side. Each orifice is of a diameter that accommodates a corresponding OD (outer dimension) size of a piece of tubing (4) or other channel. The composition of the lid may be any that can withstand the mechanical manipulation of creating the orifices.

The tubing or other channel (4) may be comprised of any material that is capable of withstanding greater than 1 Atmosphere of pressure, as well as other conditions as indicated by the desired experiment (e.g., different temperatures, different wavelengths of light, different frequencies of sound and/or vibration, different chemical compositions to which it may be exposed). The nomenclature of the channels of 4 are designated as “container inlet channel” (channel closest to the lid, in the path of arrow “A”) and “container outlet channel (channel closest to the lid, in the path of arrow” B). The composition of the container inlet channel and the container outlet channel may be the same or different.

The channels of (4) are inserted into the cap of (3) via the orifices of 5A, which are sealed from the external environment by a putty, sealant, epoxy, or the like. The material selected for the seal is dependent upon the needs of the practitioner, for example to withstand a particular experimental condition or chemical exposure. The seal material may be exclusively on the exterior of the lid, exclusively on the interior of the lid, exclusively within the opening of the lid, or any combination of the preceding.

Regardless of composition, each channel is connected to a valve (6) that permits flow and stops flow, of the inlet and/or outlet compositions, as desired by the practitioner. The valves may be the same or different, and can depend upon the types of compositions that flow through the paths indicated by the inlet arrow “A” and the outlet arrow “B”.

The composition inlet channel (7) is the conduit by which one or more composition(s) is (are) introduced via the path indicated by the arrow “A”. Any composition, or none at all, may or may not be introduced into the sample container of 1. In some aspects, the introduced composition is a gas, a liquid, a medium, a chemical, or a mixture of any of the preceding. In some aspects, ambient environmental air is allowed to flow through to the sample. In some aspects, a particular gas, such as divalent nitrogen, is provided. In some aspects, a culture medium is provided. Any amount of the introduced composition(s) may be added, up to and including the full volume available in the sample container of 1. The introduced composition may be introduced one time, a plurality of times, or continuously.

The composition outlet channel (8) is the conduit by which one or more composition(s) is (are) extracted from the sample container via the path indicated by the arrow “B”. In some aspects, a cork or other stopper is connected to the outlet valve. A universal adapter, such as a Luer-lock, may be desirable to facilitate sample extraction and maintenance of the sample container environment. The extracted composition(s) may be discarded, preserved, and/or tested.

The sample itself (9) in the container (1) may be any composition, for any application, that benefits from a controlled environment and/or requires periodic sampling for assessment.

Contemplated uses include, but are not limited to, the following: testing of plant tissue for nitrogen fixation capability; establishment of a bioreactor for cell production; microbial culture; The device can be used to collect and manipulate samples from a field satiation such as wetlands or rivers to measure changes in the composition of the headspace overtime. The device can be used to measure the effects changing the gas environments of or to any sample placed in the container.

While the invention has been particularly shown and described with reference to a preferred embodiment and various alternate embodiments, it will be understood by persons skilled in the relevant art that various changes in form and details can be made therein without departing from the spirit and scope of the invention. Various alterations, modifications, and improvements of the present disclosure that readily occur to those skilled in the art, including certain alterations, modifications, substitutions, and improvements are also part of this disclosure. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

All cited patents, patent applications, patent publications, and non-patent literature referred to in this application are herein incorporated by reference in their entirety, for all purposes, to the same extent as if each were individually and specifically incorporated by reference.

### EXAMPLES

The improved bottle apparatus were originally designed for more precise acetylene reduction assays on plants inoculated with microbes, as previous work showed that there was minimal evidence of natural fixation in designs that were aerobic. This apparatus was developed as a way to quickly purge aerobic systems to make them anaerobic. This system is a flexible, affordable tool to create different environmental systems by being able to change the atmosphere in seconds, while allowing sampling over time. Gases and/or liquids may easily be introduced, and/or extracted, while maintaining a constant intra-bottle environment so that the sample contained therein remains effectively unperturbed. Further, particular environments may be established within the controlled environment, without wasting inputs (such as anaerobic gasses).

Although specific parameters and specifications are detailed below, these are understood to be exemplary, and some deviations and alterations are contemplated to be within the scope of the disclosure.

### Example 1

### Apparatus Design

One component of the improved apparatus is a bottle that accommodates Mixed Atmospheric Gas (MAG), and was thus referred to as a “MAG” bottle.

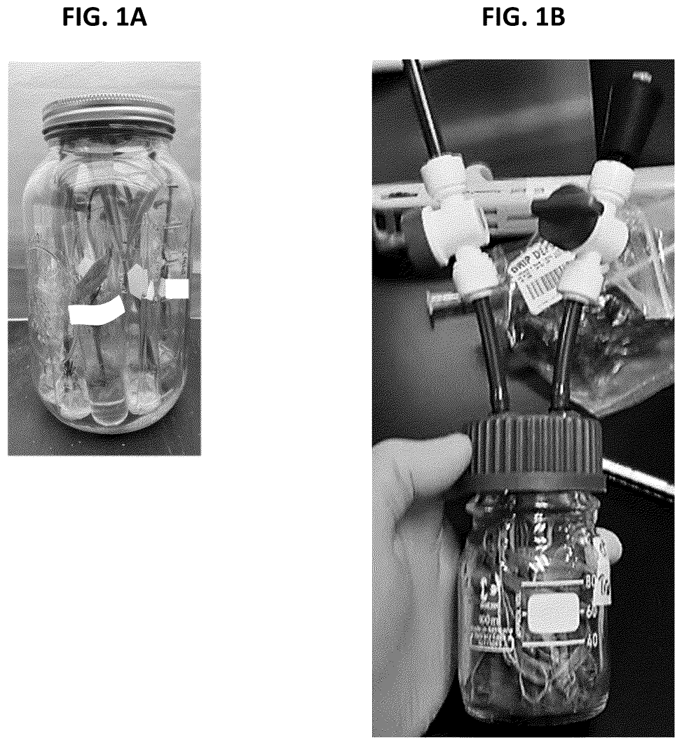

Previous designs, such as shown in FIG. 1A, were inferior, due to various reasons such as inability to maintain functional anaerobic environments, inability to easily transfer between sample collection and testing, and inability to be sterilized for re-use. Sample containers such as glass jars did not allow for easy creation of an anaerobic environment, since the movements of gas were far more cumbersome, and high pressures were not feasible.

Anaerobic sampling bottles for in planta assays were designed and tested for effectiveness in maintaining an established atmospheric system. All materials were manufactured in SAE units, and standard measurements will be referred to in the descriptions of the MAG bottle. All parts designed to hold high pressures—the system described below was tested at 20 psi and held for at least 48 hours. Maintenance of the anaerobic environment was held for up to 5 days.

Additionally, sample throughput for testing was much faster, (in one example, 24 bottles were tested in 15 minutes, vs a few hours using an anaerobic chamber), because there was no step of putting bottles into a specialized anaerobic chamber that needed to be cleaned between uses.

MAG Bottles, as exemplified in FIG. 1B, were created and tested as follows. Standard-size bottles and caps, including caps that were interchangeable between different sizes of bottles, were used for initial development. Standard, commercially-available bottles, caps, adapters, connectors, and threading are preferable, to reduce cost and improve system flexibility for a variety of applications. Each component is capable of various sterilization processes, to ensure an uncontaminated environment.
a. The bottle's cap was drilled out twice making sure to stay within central ring (as observable inside cap) using a 7/32″ or 15/64″ drill bit. The orifices were then cleaned and threaded using a′/4″ outer diameter with threads per inch equal to that of the barb that was to be installed. Alternatively, metal barb adapters may be used and will self-tap.
b. In each orifice ¼″ to ¼″ threaded barb adapter was installed.
c. Barb adapters were sealed and secured in place using a heavy-duty silicone caulk, such as RTV silicone or aquarium silicone. Barbs were verified to be sealed inside and out in order to ensure an air-tight seal, while exercising caution not to occlude the barbs opening. The silicone was allowed to set overnight at room temperature.
d. A length of ¼″ outer diameter polyethylene (food-grade) tubing was connected to each barb and ensure that they are secure. Any similar tubing may be used so long as it is semi-rigid, durable, and does not contain VOCs or rapidly degrade when exposed to a variety of pure gasses. In one example, the sections of tubing are ˜3″ long.
e. A ¼″, high pressure, inline, quick-connect ball valve was installed on each of the tubing sections. Barbed ball valves may also be used. Tapered fitting should be avoided.
f. To one of the valves, a reasonably long section of the same tubing already described was installed. This was the tubing that attached to the barb fitting of the gas regulator.
g. On the other valve, a section of tubing was installed that was just big enough to lock securely in the ball valve with enough room extra to allow for the installation of an additional ¼″ to ¼″ threaded barb adapter.
h. The barb adapter was threaded into the tubing securely.
i. A pre-drilled, or carefully drilled-out, a size 0 or similar rubber stopper with a 5/32″ orifice, was attached.
j. The needle was cut away from the hub of a 18 or 16 gauge disposable needle (carefully, using pliers and side-cutter pliers), ensuring that the needle canula remained open. The needle was safely disposed in a sharps container. Preferably, a blunt end, 16 gauge X½″ canula may be used so long as the hub fitting is a Luer-lock style. If using blunt end needles, it may not be necessary to remove or trim the needle so long as the hub fits securely when installed in the opening of the cork. (Note: original designed utilized an 18 gauge needle but then it kept breaking off. A 16 gauge ¾″ inch blunt cannula was obtained, which fit better with the spike adapter, reduced dead space, and improved ability to remove O2 contamination).
k. A hub was securely installed into the opening of the stopper, ensuring that there was an air-tight fit and that the threads of the Luer-lock were not buried in the stopper.
l. With the valve closed, a 30.0 ml syringe was attached, and an attempt was made to aspirate. It was confirmed that pressure was built in the syringe and not abated until the plunger was released. The syringe was removed from the hub and 30 ml of air was drawn. The syringe was attached to the hub and plunger depressed. It was confirmed that pressure was built in the syringe and there were no indications of gas leakage. The syringe was removed.
m. The lid was placed on an appropriate bottle.
n. A gas hose was attached to the regulator barb with the gas-hose ball valve open and tje draw port ball valve shut.
o. Gas was slowly turned on and gas pressure slowly increased from 0 to 30 PSI (=2 ATM,=1551 mmHg). No leakage was detected. The gas line ball valve was closed and the gas turned off. The gas line was removed from the regulator barb.
p. The draw port ball valve was opened, and gas was heard decompressing.

### Improvements

Additional experiments were conducted to improve upon the initial MAG bottle design. In one improvement, a two-chemical mixed epoxy is evaluated to replace the silicon, which is air- and water-proof but not as durable as epoxy (which also has the advantage of being bleach-resistant). In another improvement, part or all of the system is replaced with brass (e.g., fittings, connectors, tubing) to improve stability and reusability. In another improvement, a filter is inserted to remove potential contaminants between the bottle and the external environment.

### Example 2

### Acetylene Reduction Assay (ARA) Method

General protocols for the Acetylene Reduction Assay, prior to the improvements of the instant disclosure are given as follows.

ARA with GC-FID for Gram Positive Strains

Ensure all equipment and materials are sterilized. Wrap sealing equipment containers in foil prior to autoclaving so that they can be unwrapped in the Anaerobic chamber pass box and enter the Anaerobic chamber sterile. Seal vial openings with foil prior to sterilizing. Loose ‘seals’ are required to allow gas exchange in the pass box.
a. Prepare the anaerobic chamber by cleaning the surfaces and passing the sealing equipment through—ensure containers allow for gas exchange.
b. Add 30 mL NF 11 in 70 mL volume vial—3 reps/isolate.
c. Add 150 uL inoculant/vial which was balanced to an OD600 of 0.3 using sterile water.
d. Pass vials through Anaerobic chamber and seal under anaerobic conditions—include an empty vial (with foil ‘cap’) to add an anaerobic indicator for QC purposes.
e. Place vials in 30 C, 200 rpm for 5 hours.
f. After 5 hours, working in the fume hood, remove 10% (4mL) from the headspace of each vial and replace with the same volume of acetylene gas. NOTE: Acetylene gas is highly reactive and explosive thus the bag must be kept in the fume hood while working.
g. Incubate at 30 C, 200 rpm for 48 hrs.
h. At 48 hours (or other known timepoint), take 1mL headspace sample and place into a GC collection tube.
i. Run samples in GC using instrument method for ethylene analysis ‘split 4’ which measures acetylene peak and ethylene people
j. Amount of gas is quantified by peak area.
k. Take OD600 readings of 200 ul of the culture and TVCs of culture.
l. Analyze ethylene gas as a percentage conversion of acetylene to ethylene. This produces an estimate of total conversion.

The volume of gas produced (ethylene) can either be quantified using calibration points in Chromeleon or by calculation from the % peak area. Acetylene+Ethylene peak area % must =100% for this. From the knows amount of Acetylene added, the ethylene produced can be determined in mL. 1 M of gas=24 dm3 or 24,000 ml. Therefore 1 mM of gas=24 ml.

To calculate how many mM ethylene produced, divide amount by 24: mM ET=ml/24

To calculate RATE: mM per hour per CFU, you need to calculate mM as described above, and need to know how much of the headspace you sampled (if using calibration calculation e.g., 1 mL of headspace sampled has x mM gas but there is 6 mL headspace total so total ethylene produced=6x mM). If calculating using peak area % only the above step is not necessary, just need to know how much acetylene you added. Need to know the number of hours of incubation with Acetylene. Need to do TVCs to calculate CFU/ml then multiple your CFU value by the number of ml cultured e.g., 4mL (gneg) or 30 mL (gpos).

Rate=total ethylene mM/(time(h)×total CFU)

ARA with Oxygen Tolerance Testing Protocol

Ensure all equipment and materials are sterilized. Wrap sealing equipment containers in foil prior to autoclaving so that they can be unwrapped in the Anaerobic chamber pass box and enter the Anaerobic chamber sterile. Seal vial openings with foil prior to sterilizing. Loose ‘seals’ are required to allow gas exchange in the pass box.
a. Prepare the Anaerobic chamber by cleaning the surfaces and passing the sealing equipment through—ensure containers allow for gas exchange.
b. Take NF11 media into the Anaerobic chamber after cleaning. Add agar at 20 g/L to NF11 and place on hot plate. Briefly bring to boil to melt the agar. After melting agar, pour 30 mL of the warmed agar into 70 mL on their sides to maximize surface area to produce slants.
c. Add 150 uL inoculant/vial which was balanced to an OD600 of 0.3 using sterile water. Do so trying to maximize the surface area exposed to the inoculate.
d. Pass vials through Anaerobic chamber and seal under anaerobic conditions—include an empty vial (with foil ‘cap’) to add an anaerobic indicator for QC purposes.
e. To adjust oxygen levels, after sealing the vial, take a thin needle syringe and remove the portion of anaerobic air from the via which will be replaced with 100% pure medical grade oxygen.
f. The assay has been run at various oxygen conditions from 0% oxygen to 22% oxygen and can be increased to much higher oxygen conditions due to manual addition of oxygen. For example, to achieve 5% oxygen, remove 2.2 mL anaerobic gas by hand and add 2 mL of 100% pure oxygen back at this condition.
g. Place vials in 30 C incubator for 5 hours.
h. After 5 hours, working in the fume hood, remove 10% (4 mL) from the headspace of each vial and replace with the same volume of acetylene gas. NOTE: Acetylene gas is highly reactive and explosive thus the bag must be kept in the fume hood while working.
i. Incubate at 30 C, 200 rpm for 48 hrs.
j. At 48 hours (or other known timepoint), take 1 mL headspace sample and place into a GC collection tube.
k. Run samples in GC using instrument method for ethylene analysis ‘split 4’ which measures acetylene peak and ethylene people
l. Amount of gas is quantified by peak area.
m. Analyze ethylene gas as a percentage conversion of acetylene to ethylene. This produces an estimate of total conversion.

Volume of gas produced (ethylene) can either be quantified using calibration points in Chromeleon or by calculation from the % peak area. Acetylene+Ethylene peak area % must=100% for this. From the knows amount of Acetylene added, the ethylene produced can be determined in mL. 1M of gas=24 dm3 or 24,000 ml. Therefore, 1 mM of gas=24 ml. To calculate how many mM ethylene produced, divide amount by 24: mM ET=ml/24.

To calculate RATE: mM per hour per CFU, need to calculate mM as described above. Need to know how much of the headspace you sampled (if using calibration calculation e.g. 1 mL of headspace sampled has x mM gas but there is 6 mL headspace total so total ethylene produced=6x mM). If calculating using peak area % only the above step is not necessary — just need to know how much acetylene you added. Need to know the number of hours of incubation with Acetylene. Need to do TVCs to calculate CFU/ml then multiple your CFU value by the number of ml cultured e.g., 4 mL (gneg) or 30 mL (gpos).

Rate=total ethylene mM/(time(h)×total CFU)

ARA Testing of Plants with Standard Apparatus

Purpose: Develop a positive control for ARA In-Planta Assay using Soybean and a known bradyrhizobium colonizer

Bradyrhizobium japonicum was inoculated on R2A media. After 3 days of growth, a small lawn and very tiny colonies were formed. These were transferred to three separate spread plates on R2A.

Eight mason jars were prepared and confirmed to hold gas.

Bradyrhizobium was then spread on soybean roots, and these were placed in a growth chamber. Although it can take ˜18 days after germination to form n-fixing nodules, many plants are unhappy in phytagel growth. Because of this, it was decided to test for ARA starting after 6 days, at which time there was clear evidence of growth of bacteria. Plants were transferred into mason jars that same day, and 190 mL of acetylene was added.

Plants were grown in two tubes with 5 plants in each tube, and sampled at 24 h & 48 h.

Although it was shown that the mason jars were able to hold samples in an air-tight matter if not disturbed or opened, the ARA results showed only a trace level of activity where ethylene is detected on the GC. Repeated tests with other plant species confirmed that minimal acetylene reduction was able to be detected using this standard, but inferior, method.

### Example 3

### ARA with the Improved Apparatus of the Instant Disclosure

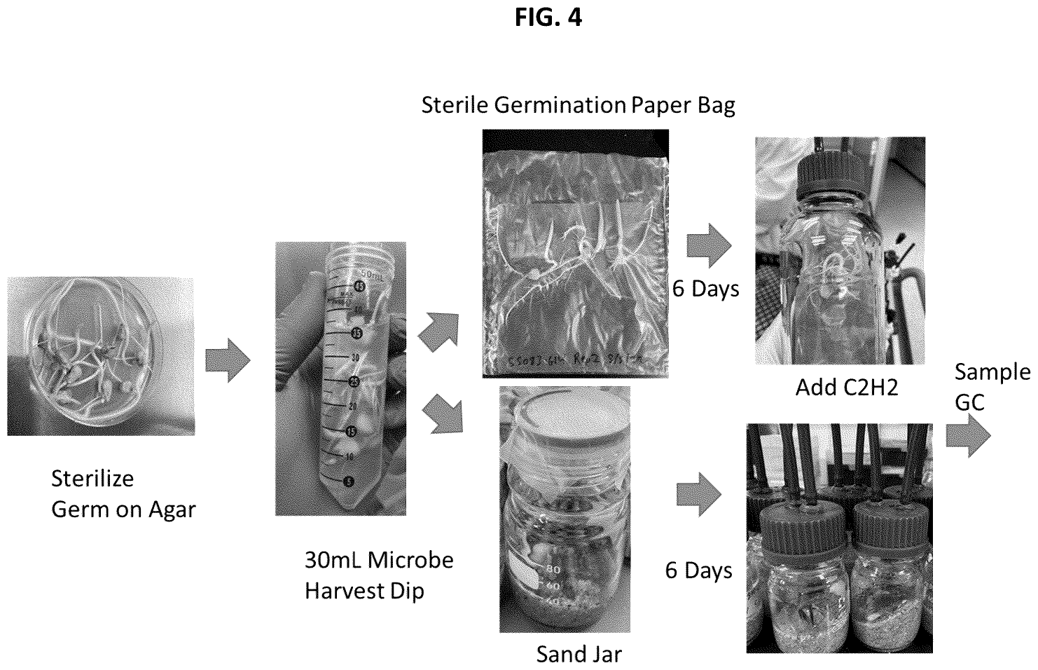

Following the construction and pressure testing of the newly-developed MAG Bottles, 4 untreated plant samples were selected and transferred with ˜50% of the root-associated soil to 1 bottle each. The general methodology is shown in the pictures of FIG. 2. Sample preparation for different inputs (germination in a paper bag enclosed in a plastic bag, germination in a sand jar) is shown in FIG. 4.
a. Samples were taken to the lab where 2.0 ml of RO-H2O was added to maintain root and microbial viability.
b. Bottles were sealed and both ball valves were opened. Each system was purged using mixed anaerobic gas for 5 seconds with a gas pressure of 16 PSI. This is equivalent to 1.1 ATM compared to standard pressure at sea level of 1.0 ATM. The draw port valve was closed while gas was running, resulting in ˜0.1 ATM overpressure.
c. The gas line valve was closed and disconnected from regulator.
d. This process was repeated for each sample.
e. Samples were incubated @room temp for 5 hours before 10% C2H2 was injected through draw port.
f. 1.0 ml of headspace was collected from the draw port @24, 48, and 96 hours.
g. Samples were run through GC.

ARA results showed that the improved MAG bottles were able to maintain atmosphere as established, and showed no acetylene conversion to ethylene, which was expected as the plants were untreated with nitrogen-fixing microbes, and any conversion would be inherent within the plant itself.

### Example 4

### ARA with the Improved Apparatus of the Instant Disclosure

Field sampling bottles were designed and created to allow for anaerobic assays to be conducted on plant samples. For this trial run, the effectiveness of these bottles, were tested using barrier-plant samples harvested from UC Davis test plots where field trials were being conducted. As with the standard ARA protocol, 10% of the system headspace was replaced with acetylene gas. Gas samples were then be collected and transferred top GC collection vials @0, 24, 48, and 96 hours. Samples were run in GC per SOP.

Two growth setups were prepared: (1) placing inoculated seedlings into an open plastic bag with germination paper, and (2) placing inoculated seedlings into jars with a sand medium. It was hypothesized that an in planta system would more accurately measure direct Nitrogen fixation than a culture tube based system.

Inoculation with microbes was performed, for example by submerging newly-germinated seedlings in a microbial suspension for 20 min.

Materials: Germination Paper, Autoclaved open plastic bags, Corn Seeds, Inoculum (microbial suspension), water negative control.

### Seed Preparation and Germination

### Surface Sterilize Corn Seeds

a. Place corn seeds into large glass beaker on spin plate.
b. Add 70% ethanol and stir for 1 minute
c. Decant ethanol, catching any seeds in a sterile ceramic filter and knocking back into beaker.
d. Add 0.5% sodium hypochlorite (10% bleach) and stir for 5 minutes.
e. Decant bleach
f. Rinse 4 times with RO water
g. Place sterilized corn seeds on agar 0.9% in dark at 25 C for 4 days
h. To test for sterility, a few seeds were placed and shaken on a plate of R2A media
i. Prepare N&C Free Rooting Solution
j. NF11 base media without glucose. This was comparable to other nutrient systems published previously.

### Prepare Inoculum (Day Of)

a. Grow up selected microbes and harvest from an R2A plate to a 50 mL centrifuge tube, using 1.3 mL of sterile RO water and an L-spreader.
b. Add sterile RO water up to the 35 mL mark
c. Take OD600 reading
d. Place up to 15 germinated seedlings into each inoculum, careful not to crush/break roots or hypocotyl, for 20 min
e. Place up to 15 germinated seedlings into each inoculum, careful not to crush/break roots or hypocotyl, for 20 min

### Germination Paper Method

a. Autoclave germination paper inside open plastic bags ahead of time. Dry completely.
b. Add 15 mL of NF11—Carbon free rooting solution to each bag to fully saturate germination paper
c. Using sterile tweezers transfer 3 seedlings per rep of each sample to sterile germination paper pouch, placing no deeper that 1-2 inches from the opening of the bag
d. Position seedlings so that their hypocotyl is pointing upwards
e. Gently compress bag between to pieces of cardboard and place standing upright in growth room
f. Cardboard will diminish light exposure to seedling roots and will keep seedlings from sliding down deeper into the bag

### Sand Jar Method

a. Fill 100 mL Schott bottles (the true volume is 130 mL) with 50 g of industrial grade sand and a few mL of RO water. Cap bottles with foil and autoclave
b. Add 15 mL of NF11—Carbon free as nutrient source
c. Using sterile tweezers place 1 seedling per jar, positioning it so the hypocotyl is pointing upwards. Cover roots with sand.
d. Cap with parafilm to retain moisture, but allow light and gas exchange. Place in growth room

Not all seedlings had uniform exposure to inoculum because of centrifuge tube method requiring careful placement and removal of seedlings one by one. A contemplated improvement is using tea strainers to address this.

All work was done in biosafety cabinet. Aside from isolate harvest, majority of this work can be done in the flowhood.

Seedlings roots had grown longer than desirable by day 4, moving forward this timeframe may be reduced to two or three days. Seedlings did well in the dark in 25 C.

### Anaerobic Condition Induction and Addition of Acetylene

If desired, prior to adding acetylene a 200 mL or greater syringe should by attached to draw/injection port, and stopcock should be opened to allow for decompression of system. This will be needed in order to maintain atmosphere just slightly more than 1.0 ATM after the addition of C2H2.

Sand jars- Parafilm was removed from jar opening and modified MAG bottle caps were put in place. With outlet open, inlet of bottle cap was attached to (5% hydrogen 80% nitrogen 15% carbon dioxide) gas tank and flooded to expel and replace oxygen. Outlet closed before removing from tank. 10 mL of Acetylene was added via syringe (without needle) into the inlet and bottles were placed back in grow room.

Bags- Bags were loosely rolled in order to be placed inside 500 mL bottles. Modified MAG bottle caps were put in place. With outlet open, inlet of bottle cap was attached to (5% hydrogen 80% nitrogen 15% carbon dioxide) gas tank and flooded to expel and replace oxygen. Outlet closed before removing from tank. 50 mL Acetylene was added via syringe (without needle) into the inlet and bottles were placed back in grow room. Data Collection

Large bottles, containing the rolled up bags, had 20 mL of gas sampled. 20 mL of headspace was removed from the gas chromatograph tubes before inserting sample.

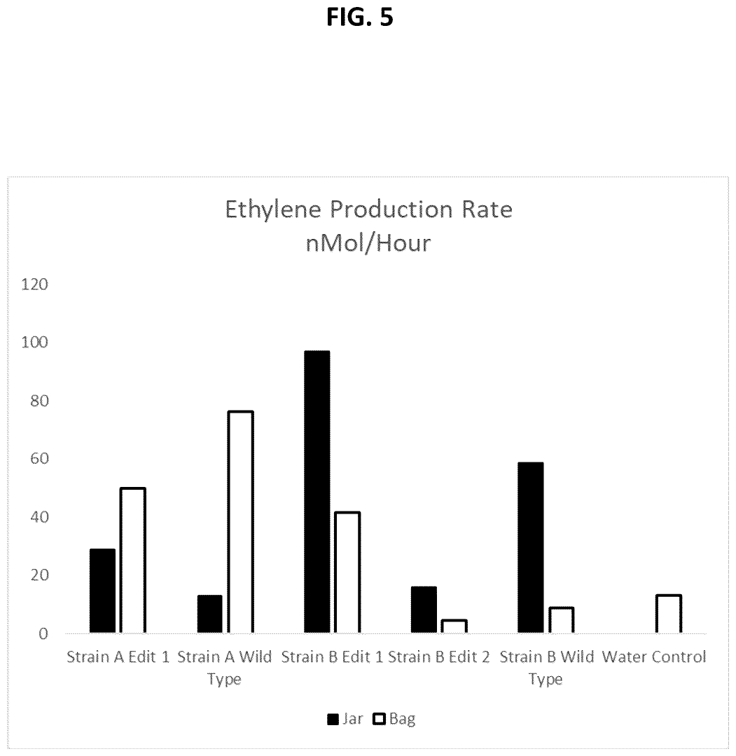

Sand jars had 5 mL of gas sampled. 5 mL of headspace was removed from the gas chromatograph tubes before inserting sample. Samples were collected at 25, 48, and 72 hours after acetylene addition. Ethylene production results at the 48 hour timepoint are shown in FIG. 5.

It is claimed:

1. An apparatus for the establishment, maintenance, and optional testing of a sample in a controlled environment, the apparatus comprising:
(a) a sample container;
(b) a lid that attaches to the sample container, wherein the lid has two orifices;
(c) a chamber inlet channel inserted into one of the two orifices and a chamber outlet channel inserted into the other of the two orifices;
(d) a sealant material in or around each of the two orifices into which the inlet channel and outlet channel have been inserted;
(e) an inlet valve connecting to the chamber inlet channel and a composition inlet channel;
(f) an outlet valve connecting to the chamber outlet channel and a composition outlet channel;
(g) a composition inlet channel;
(h) a composition outlet channel; and
(i) a sample.

2. The apparatus of claim 1, wherein the sample container and lid are synergistically threaded.

3. The apparatus of claim 1, wherein the sample container is substantially made of glass.

4. The apparatus of claim 1, wherein the sealant material comprises silicone or epoxy.

5. The apparatus of claim 1, wherein the inlet valve and/or the outlet valve are of ballcock design.

6. The apparatus of claim 1, wherein the composition inlet channel is connected to a source of a gas.

7. The apparatus of claim 1, wherein the composition inlet channel is connected to a source of a liquid.

8. The apparatus of claim 1, wherein the sample comprises a living organism or part thereof.

9. The apparatus of claim 8, wherein the sample comprises a plant.

8. The apparatus of claim 8, wherein the sample comprises a microbe.

11. The apparatus of claim 8, wherein the sample comprises a plurality of living organisms or parts thereof.

12. The apparatus of claim 8, wherein the sample comprises at least one plant and at least one microbe.

13. The apparatus of claim 1, wherein the composition outlet channel is connected to a syringe.

14. The plurality of apparatus of claim 13, wherein the apparatus are arranged in series.

15. The plurality of apparatus of claim 13, wherein the apparatus are arranged in parallel.
